(19) United States
(12) Patent Application Publication
(10) Pub. No.: US 2007/0179805 A1
(43) Pub. Date: Aug. 2, 2007

(54) Method and system for improving the quality of service and care in a healthcare organization

(76) Inventors: Richard L. Gilbert, Charlotte, NC (US); Brent P. Holway, Charlotte, NC (US); Joseph P. Coyle, Cornelius, NC (US); Charles Dana Hershey, Charlotte, NC (US); Janet E. Beck, Belmont, NC (US)

Correspondence Address: NELSON MULLINS RILEY & SCARBOROUGH, LLP, 1320 MAIN STREET, 17TH FLOOR, COLUMBIA, SC, 29201, US

(21) Appl. No.: 11/342,143

(22) Filed: Jan. 27, 2006

(51) Int. Cl. G06Q 10/00; A61B 5/00

(52) U.S. Cl. 705/2; 600/300

(57) ABSTRACT

A method for improving the quality of healthcare, efficiency, and patient satisfaction procedures is provided. The method includes conducting at least a first survey of patients regarding care provided before, during, and after a procedure, wherein the survey focuses on benchmarks identified as being relevant to quality of care. The method further includes entering the results of the first survey into a database configured to store the results, reviewing the results of the first survey to determine the quality of care provided during the procedure, identifying incidences of poor quality of care, and comparing the results of the first survey with results of surveys taken from other patients to recognize patterns of poor quality of care. The method further includes addressing the incidences of poor quality of care by developing methods to address the recognized incidences of poor quality of care.

### BACKGROUND OF THE INVENTION

The invention relates to the field of quality improvement. More specifically, the invention relates to the field of quality improvement in the healthcare industry.

Numerous studies have highlighted the high rate of medical errors and the need for fundamental changes in the healthcare delivery system to eliminate gaps in quality.

Healthcare facilities such as hospitals and clinics are generally organized in departments specializing in specific areas of medical science such as immunology, cardiology, and radiology. Generally, specialized personnel and equipment are available in each department to provide medical treatment in the area of specialization. Often times, these departments must repeatedly perform the same or similar procedures on many patients.

The competence and efficiency with which each of these tasks is conducted affects the overall quality and efficiency of the healthcare organization. It also affects the patient's safety and satisfaction with the services performed. Thus, to the extent that efficiency and satisfaction could be improved, the operation of the organization, including such things as quality and profitability, could also likely be improved.

Previous methods of improving the quality of healthcare procedures have focused on statistical sampling of patients and procedures. For example, some healthcare organizations examine patient charts on a monthly basis and look for incidences of poor quality of care. This method of analyzing quality often includes “surveying” the charts, i.e., examining a random sampling of charts. Accordingly, there is a time delay between the incidence of poor quality and the discovery of the poor quality. This time delay may lead to multiple incidences of poor quality before the discovery is made. Additionally, these problems may not be recorded on the charts and may be forgotten by the time the review is conducted.

Another drawback to this method is random chart sampling. Many quality problems occur only at a very small scale and would therefore be missed by random chart sampling. A healthcare organization would, therefore, be unlikely to develop a thorough understanding of the quality issues within the organization by utilizing this method.

Moreover, the various healthcare professionals that may fill out a patient's chart while that person is undergoing treatment are rarely trained to observe quality indicators specific to a desired area of healthcare. For example, operating room nurses may not be trained to observe post-anesthesia issues and may not, therefore, note those issues on a chart. By the time a periodic review of charts occurs, any post-anesthesia issues relating to the quality of care would remain undiscovered.

Another previous method of monitoring and improving the quality of procedures in a healthcare organization includes analyzing billing data to determine whether any incidences of poor quality occurred during a patient's treatment and care. This method often results in inaccurate data due to the challenges inherent in using billing data. For example, there is a significant time delay between a procedure and the billing for the procedure. Additionally, billing data is an unreliable source of quality information, because not all quality indicators are reflected in the billing data.

Many insurance companies and hospitals, as well as Medicare and Medicaid, are requiring healthcare organizations to negotiate medical costs. In order to participate in insurance programs, Medicare, and Medicaid, many healthcare organizations are required to reduce their costs for services as a result of this required negotiation process.

One form of negotiation that is being implemented and encouraged by these groups is the pay-for-performance method of negotiation. Pay-for-performance seeks to introduce traditional market forces to motivate healthcare organizations' adherence to evidence-based practices. The goal is to reward healthcare efficiency and effectiveness through monitoring and reporting on treatment patterns and corresponding health outcomes.

Pay-for performance has the potential to re-align the incentives of all the major stakeholders in the healthcare marketplace. For example, employers, health plans, and government purchasers who can reduce the cost of their benefits programs while improving service quality and the outcomes of individuals enrolled in their plans will likely benefit from the introduction of pay-for-performance programs. Providers who are rewarded for delivering higher quality care and provided increased payments which can be used to invest in systems that further improve patient management and compliance with practice guidelines, as well as consumers who are provided better information for selecting the highest quality providers resulting in higher levels of satisfaction and improved outcomes also benefit from pay-for-performance programs.

The initiation of a pay-for-performance environment in health care has been driven principally by multiple industry studies focused on medication errors and patient safety. There is general agreement that quality of care is not advancing as quickly as it should. Additionally, there are wide geographic regional differences in the amount and quality of care provided. Past attempts at improving quality by publicly releasing hospital morbidity data and improving consumer choice have had suboptimal effects. This is likely because the healthcare organizations are unable to form a clear, complete, and accurate report on the quality of care provided.

Additionally, even when the quality statistics are available, they are often provided by the random sampling method or billing data review method previously discussed. Not only do these methods provide a less accurate understanding of the quality levels as discussed above, they also do not fully trace or identify the source of the quality issues (i.e., practitioner performance vs. process related quality issues). By not understanding the source of the quality issues, the healthcare organization may be unable to fully address and correct those issues. If this situation is present under a pay-for-performance environment, the healthcare organization may not be able to charge the fees necessary to recoup their costs.

### SUMMARY OF THE INVENTION

In one aspect the invention is a method for improving the quality of healthcare procedures. The method includes conducting at least a first survey of patients regarding care provided before, during, and after a procedure, wherein the survey focuses on benchmarks identified as being relevant to quality of care. The method further includes entering the results of the first survey into a database configured to store the results, reviewing the results of the first survey to determine the quality of care provided during the procedure, identifying incidences of poor quality of care, and comparing the results of the first survey with results of surveys taken from other patients to recognize patterns of poor quality of care. The method further includes addressing the incidences of poor quality of care by developing methods to address the recognized incidences of poor quality of care.

In another aspect, the invention is a system for improving the quality of healthcare procedures. The system includes at least one questionnaire, including a list of indicators recognized as relating to the quality of healthcare, to be answered by patients undergoing a medical procedure, a database for storing the answers provided on the at least one questionnaire, a review process for recognizing incidences of poor quality of care before, during, and/or after a procedure based on the answers to the at least one questionnaire. The system further includes a quality control process that recognizes patterns of poor quality of healthcare and institutes corrections in response to said recognized patterns.

In yet another aspect, the invention is a method of designing a pay-for-performance valuation for a healthcare organization.

The foregoing, as well as other objectives and advantages of the invention and the manner in which the same are accomplished, is further discussed within the following drawings and detailed description.

### BRIEF DESCRIPTION OF THE DRAWINGS

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements.

FIG. 1 is a flow chart representing various stages of indicator input in accordance with the present invention.

FIG. 2 is a flow chart representing one embodiment of the present method.

### DETAILED DESCRIPTION

The invention relates to a method and system for improving the quality of healthcare. More specifically, the invention relates to a method and system for improving the quality of the procedures of a healthcare organization by providing timely and relevant feedback. In another embodiment, the invention relates to a method of determining a pay for performance valuation based on the quality provided by a healthcare organization.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In one aspect, the invention is a method for improving the quality of healthcare procedures. For ease of discussion, the method will be described with reference to anesthesia care. Those having ordinary skill in the art will recognize that the invention is applicable to healthcare processes and procedures other than anesthesia care, such as, but not limited to, oncology, surgery, and the like for which quality control can be important to improve services, patient satisfaction, economics, and the like. Accordingly, the description shall not be limited to anesthesia care.

The method includes conducting at least a first survey of a plurality patients in a healthcare organization, regarding care provided before, during, and after a procedure. Stated differently, the method includes collecting data and assimilating the data in a form suitable for analysis, review, etc., to determine if any incidences of poor quality are present. An exemplary survey may focus on benchmarks, or indicators, identified as being relevant to quality of care. For example, in a survey relating to anesthesia care, the survey may include indicators relating to the quality of anesthesia care as recognized by various professional organizations such as but not limited to the American Society of Anesthesiologists, the Institute of Medicine, and the Joint Commission for Accreditation of Healthcare Organizations (JCAHO).

In an exemplary embodiment, the survey is completed by an individual trained to recognize the existence of relevant indicators. In an exemplary embodiment, the survey may be conducted by one or more individuals including, but not limited to, trained medical personnel such as a nurse or doctor. The individual can conduct the survey using any suitable means of data collection such as but not limited to studying a patient's condition, reviewing a patient's chart, conversing with the patient to ascertain the patient's views regarding such issues as pain care, and the like. In another exemplary embodiment, the individual completing the survey discusses the indicators with the patient. In yet another embodiment, the individual completing the survey combines the two previously discussed methods. Other methods known in the art for completing the survey are also contemplated as useful in accordance with the present invention.

In one embodiment, the survey may be completed in one session. In another embodiment, the survey may be completed at different stages throughout the continuum of care of the healthcare procedure. For example, the survey may be completed during one or more of patient preoperative holding, the operating room, the post anesthesia recovery room, the post operative home floor, combinations thereof, and the like. In yet another embodiment, a different survey may be completed at each stage of the healthcare procedure. In another embodiment, a combination of the above completion strategies may be employed.

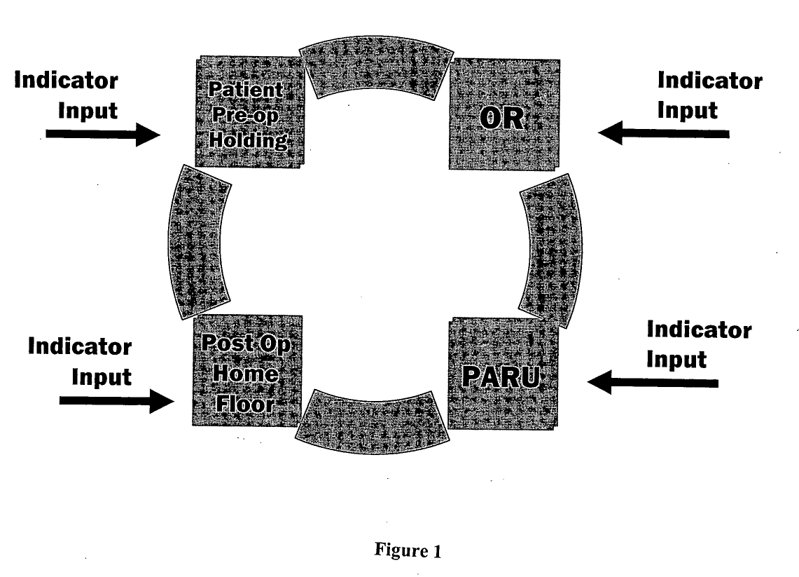

FIG. 1 is a representative flow chart illustrating nonlimiting examples of entry of responses onto a survey throughout the continuum of care in accordance with the present invention. As demonstrated by the figure, indicators may be recognized at various stages of a procedure. For example, indicators may be recognized during patient preoperative holding, in the operating room, in the post anesthesia recovery unit (PARU), and/or on the post operative home floor. In the example depicted in FIG. 1, the presence of the relevant indicators are noted on the survey at each stage of the procedure, as they occur. This enables timely recognition of quality issues, and insures better accuracy in the quality process because there is little to no time delay between occurrence of an incidence and recordation of the incidence. This lack of a significant time delay helps prevent oversight (or forgetfulness) that may lead to failure to note relevant incidences when surveys are completed at later stages, such as in processes previously discussed including, but not limited to, the random chart sampling and the billing data methods of review.

Those having ordinary skill in the art will recognize that incidences of poor quality or other indicators may occur at other stages of a procedure than those depicted in FIG. 1. FIG. 1 shall not, therefore limit the present method to recognition and recordation of indicators at the depicted stages.

With reference to surveys relating to the quality of anesthesia care, exemplary surveys may request information on more than 30 indicators. The surveys may request information on more than 40 indicators. The surveys may also request information on more than 50 indicators. Generally, the survey may include 10, 20, 30, 40, 50, or more indicators. Those having ordinary skill in the art will recognize that a larger number of indicators may result in more accuracy and better monitoring of the quality of care provided by the healthcare organization.

Those having ordinary skill in the art will recognize that the survey may include fewer or more of the standard recognized indicators and may further include standards developed by the healthcare organization. Moreover, indicators developed by medical professionals and/or associations in disciplines other than anesthesia care may be included on the survey depending upon the type of procedure being evaluated.

The survey may be completed in a traditional paper format. Alternatively, the survey may be completed with the use of a computer-based program, for example on a hand-held computer device, a laptop computer, and/or over the internet. In one embodiment, a combination of a paper based survey and a computer-based survey may be completed.

It may be desirable to complete portions of the survey relating to post-procedure indicators after a patient has left the healthcare organization. In this embodiment, the survey may be completed over the Internet, over the telephone, by mail, by personal visit with the patient, combinations thereof, and the like. For example, a patient satisfaction survey may be conducted after completion of the healthcare procedure.

In an exemplary embodiment, substantially all of the patients in a healthcare organization are surveyed. Stated differently, at least about 50%, more preferably at least about 75%, of the patients of the healthcare organization participate in the survey. In one embodiment, every patient of the healthcare organization participates in the survey. Those having ordinary skill in the art will recognize that the percentage of patients included in the surveys may be most accurately measured from the time the method of the present invention is implemented.

After completion of the survey, the present method may include a review process. In an exemplary embodiment, the results of the survey are available to the individual practitioner to judge the quality of care provided and to identify potential areas of poor quality. In one embodiment, the results of the survey are available on a real-time basis. In another exemplary embodiment, the results are available to the practitioner within about twenty four hours. In yet another embodiment, the results are available to the individual practitioner within 48 hours. Those having ordinary skill in the art will recognize the benefits provided by timely accessibility of the results of the survey. For example, a practitioner with timely access to the results may be able to recognize a quality issue and begin the process of correcting that issue immediately. When quality is measured retroactively, as in the random sampling method or the billing method discussed above, the time between the incidence of poor quality and the recognition of existence of the incidence of poor quality may be sufficient to allow repeated incidences to occur.

As a part of the review process, the results of the surveys given to the patients of a healthcare organization are compared. The comparison step may be conducted manually or by software equipped with character recognition capabilities. As a result of this comparison, repeated incidences of poor quality of care may be identified. In an exemplary process, a committee including medical personnel trained in the relevant discipline may review and compare the surveys. In another exemplary process, at least one physician and one nurse review and compare the surveys.

In another exemplary embodiment, the committee may identify incidences of poor quality of care by several criteria. For example, a particular facility may be recognized as having repeated incidences of poor quality. In another embodiment, a particular physician may be recognized as having repeated incidences of poor quality of care. In yet another embodiment, certain procedures may show repeated incidences of poor quality of care. Those having ordinary skill in the art will recognize that these criteria, as well as other criteria known in the art, may not be mutually exclusive. The specification and claims shall be understood to include combinations of these criteria as well as incidences wherein only a single criterion is indicated.

In one embodiment the present method includes an additional reviewing step, wherein the results of the surveys taken from patients are reviewed by a quality control committee charged with recognizing incidences of repeated poor quality. This additional review step, when employed, may serve as a backup quality step to ensure that repeated incidences of poor quality of care are recognized and addressed.

In each of the above review processes, the surveys of patients at different facilities may be compared. Additionally, surveys of patients having different physicians and other personnel may also be compared. Those having ordinary skill in the art will recognize that other comparison criteria may be utilized as a part of each of the above review processes. The specification and claims shall be read and understood to include these other known criteria.

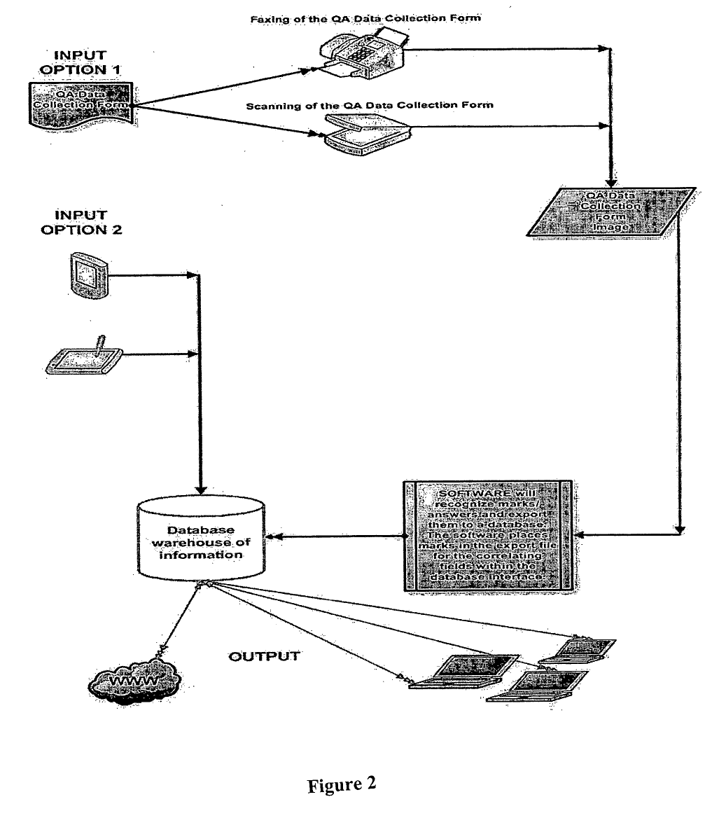

FIG. 2 is a representative flow chart of the data collection and storage steps of the present method. As depicted in FIG. 2, responses to the survey may be collected on a data collection form, for example a paper form, that may then be faxed or scanned into a database warehouse of information. Alternatively, responses to the survey may be collected on a handheld computer device or laptop and then downloaded into the database. Information may then be available to individuals having access to the database. FIG. 2, in combination with the previously discussed FIG. 1, demonstrate the timeliness of the entry of data and availability of the data to medical personnel, the healthcare organization, and other interested parties.

Those having ordinary skill in the art will recognize that other methods of input of data and availability of data than those depicted in FIG. 2 may be available. FIG. 2 shall not, therefore limit the present method to the depicted data entry and availability methods.

The review processes described above may be utilized to identify facilities, medical personnel, procedures, techniques, and the like that demonstrate repeated incidences of poor quality of care.

Many healthcare organizations are members of associations of organizations that provide certain classes of healthcare. For example, the American Society of Anesthesiologists (ASA) is an association of anesthesia care providers. These associations often identify benchmarks, or goals, that their members strive to achieve. Alternatively, the associations may publish national averages of incidence occurrence. For example, the ASA has published a national benchmark for medication errors of 5.26% and aspiration incidences of 0.3%. By using the above described processes, anesthesia care providers may determine how their healthcare compares to the healthcare provided nationally.

Moreover, by surveying a larger number of patients in a more timely manner than previously utilized processes, the present process may result in a more accurate determination of the number of incidences of poor quality of care.

In another aspect, the invention is a system for improving the quality of healthcare procedures. In one embodiment, the system includes at least one questionnaire, or survey, including a list of indicators recognized as relating to the quality of healthcare. In an exemplary embodiment, the questionnaire relates to a patient's specific experiences before, during, and after a procedure.

In one embodiment, the present method includes classifying the indicators into various broad categories. For example, the specific indicators may provide information about one or more of patient satisfaction, efficiency, practitioner performance, and/or the overall process performance. It may be desirable to group the indicators according to these (or other) categories to better enable processing of the results and to better understand the quality issues and their root causes.

Those having ordinary skill in the art will recognize that many of the indicators are those commonly recognized by the various associations described above. Similarly, many of the indicators included in the questionnaire were recognized and developed by the inventors in accordance with the present invention.

In one embodiment, the indicators may be one or more of case cancellation indicators, case delay indicators, airway and respiratory system indicators, cardiovascular indicators, medication related indicators, neurological block related indicators, critical incident indicators, post-operative indicators, combinations thereof, and the like. In an exemplary embodiment, the indicators may reflect one or more of practitioner performance, group operations, group procedures, group systems issues for process performance improvement, combinations thereof, and the like. Those having ordinary skill in the art will recognize that the list of indicators is representative only and is not intended to be exhaustive. Other indicators recognized by those having ordinary skill in the art are also contemplated as useful in accordance with the present invention.

Examples of exemplary case cancellation indicators and their definitions include one or more of pre-operative order violations, abnormal EKG, abnormal labs, combinations thereof, and the like.

Examples of exemplary case delay indicators are one or more of pre-operative order violations, delayed surgeon, delayed anesthesiologists, operating room turnover delay, unavailable or delayed lab results, unavailable or delayed x-rays, combinations thereof, and the like.

Exemplary airway and respiratory system indicators may be one or more of difficult intubations, failed intubation, aspiration, laryngospasm, dental damage or loss, persistent hypoxeia, bronchospasm, ventilator induced injury, sleep apnea, combinations thereof, and the like.

In one embodiment, exemplary cardiovascular indicators may be one or more of EKG changes, changes to blood pressure, perioperative beta blocker protocols used in at-risk patients, combinations thereof, and the like.

Exemplary medication related indicators may be one or more of prolonged NM block, the use of an antagonist, medication errors, unplanned administration of an antibiotic after an incision is made, combinations thereof, and the like.

Examples of exemplary neurological block related indicators may be one or more of wet tap, spinal requiring mechanical vent and/or intubation, failed labor regional, failed operating regional, nerve injury, peripheral nerve injury, combinations thereof, and the like.

Examples of exemplary critical incident indicators may be one or more of death, anaphylaxis, aspiration, perioperative bronchospasm, laryngospasm, perioperative hypoxemia, cardiac arrest, blood pressure changes, EKG changes with evidence of ischemia or dysrhythmia required, extended post-anesthesia care unit stay, failed airway, heart attack, pulmonary edema, unanticipated admission to ICU, unplanned reintubation, stroke, wrong site procedure, medication error, transfusion error, nerve injury from position, post-dural headache, falls, combinations thereof, and the like.

Examples of exemplary post-operative indicators may be one or more of hypothermia, post-operative nausea, post-operative vomiting, problems with pain control, prolonged regional block, prolonged stay in the post-anesthesia care unit, urinary retention, post-dural puncture headache, awareness under general anesthesia, intra-operative normothermia in colorectal surgery, for patients with diabetes melatis: glucose measured and sliding scale insulin protocol implemented for blood sugar greater than 200, implementation of head of bed 30° in mechanically ventilated patients, combinations thereof, and the like.

Examples of exemplary additional indicators may be selected from one or more of eye problems, burn injury, pressure injury, medication or fluid extravasations, line complication with central venous line, pneumothorax, hemothorax, hydrothorax, falls, equipment problems, fire, regionals performed on the wrong location, combinations thereof, and the like.

Those having ordinary skill in the art will recognize that the above indicators are intended to be representative rather than exhaustive. Accordingly, other indicators known in the art are contemplated as useful in accordance with the present invention. The specification shall be, therefore, read and understood to include other indicators known to those having ordinary skill in the art.

In one embodiment, the present system may also include a definition worksheet to ensure consistency in the completion of the questionnaires. For example, indicators such as those described above may be defined differently by different medical personnel. In the present embodiment, a definition guide may be provided to eliminate the possibility of different responses based on the personnel completing the questionnaire. This will further aid a healthcare organization in determining the level of quality being provided because the healthcare organization may be assured that the information provided is being provided in a consistent manner.

By developing a more accurate understanding of the present level of quality within an organization, the members of that organization will be better able to assess the quality level and determine steps that will help improve the level of quality within the organization where needed. Moreover, the organization may be able to utilize this better understanding of their quality to attract more patients to the organization. Additionally, evidence of a high level of quality within a healthcare organization may aid the organization in attracting top physicians and other healthcare providers to the organization, thereby further improving the quality being provided to patients.

The present system may further include at least one database for storing the information provided on the at least one questionnaire. The information may be entered into the database by scanning a paper form, faxing a paper form into the database from a remote location, by manually entering the information on the questionnaire, or automatically, for example, where the questionnaire was completed on a hand-held computer device, the information may be automatically downloaded into the database.

In an exemplary embodiment, the database may further include a software program having character recognition capabilities. An exemplary software program in accordance with this embodiment may recognize responses on the previously described questionnaire. In one embodiment, the software program may recognize a response and flag the questionnaire for further review as a result of the presence of that response. Stated differently, the software may include an “if this, then that” feature that is capable of organizing questionnaires into categories based on the responses given on the questionnaires.

Exemplary software programs in accordance with the present invention may also include the capability to search and review the questionnaires by various criteria, including, but not limited to date, patient, physician, operating room personnel, facility, combinations thereof, and the like. This searching ability may serve to further streamline a review process and further aid the healthcare organization in monitoring the quality of their services. This searching ability may be achieved through the use of software programs including character recognition capabilities.

In one embodiment, the database will be based on the World Wide Web (i.e., the Internet) to allow use of and access to the database from remote locations, such as different facilities from which the healthcare organization operates. In this embodiment, the database may be appropriately encrypted to ensure patient and physician confidentiality. In another embodiment, the database may be based in a server, to which the members of the healthcare organization have access such as, for example, over an intranet.

The present system further includes means for reviewing the responses to recognize incidences of poor quality of care in a timely manner. In one embodiment, the means may include a review process for timely recognition of incidences of poor quality of care occurring before, during, or after a procedure. In another embodiment, the means may include software implemented recognition. The review process may include studying the responses on the previously described questionnaire to determine the level of quality provided as a part of a healthcare procedure. In an exemplary embodiment, the review process includes a review committee including medical personnel trained in the relevant discipline. In another exemplary embodiment, the review committee may include at least one physician and one nurse that are trained to review the questionnaires and recognize indicators of poor quality.

The present system may further include means for controlling quality of care that recognize patterns of poor quality of healthcare based on the responses on the questionnaire and institutes corrections in response to said recognized patterns. In one embodiment, the means may include a quality control process that recognizes patterns of poor quality of healthcare and institutes corrections in response to the recognized patterns. For example, the previously described committee may recognize a pattern of case delays based on the responses to the questionnaire. If such a pattern were recognized the committee may then research further to determine the cause of the high level of case delays. A review of substantially all of the questionnaires showing case delays may show that the case delays are most often due to a physician arriving late.

Once the committee has determined the cause of the case delays (in this example, late arrival by a physician), the committee may discover that it is a single physician that is demonstrating a pattern of late arrival. Late arrival on the part of a physician may delay the immediate healthcare procedure for which the physician is tardy. It may also delay procedures later in the day because the operating room may not be available on schedule due to the late start of the earlier procedure. An additional benefit of the present system and method is the timeliness of the feedback. For example, the late arrival problems may be discovered earlier than would be possible in the previous methods of measuring quality, allowing the problems to be corrected in a more timely fashion.

Once the source of the case delays has been identified, the committee may determine the appropriate action to rectify the issue. For example, the committee may be able to address the situation directly to the physician. Because the organization will have developed such comprehensive quality information through the use of the present system and process, the committee may be able to demonstrate the domino effect on quality caused by the physician's tardiness to a scheduled procedure. With this information, the problems caused by the delay may be more easily understood and the physician may be willing to address the situation himself without further action from the committee.

If, however, the committee were to determine that the physician was not addressing the situation personally, the committee could put into effect other incentives for on-time arrival, such as docked pay for late arrival, etc.

Those having ordinary skill in the art will recognize that the above example is representative of the use of the present system and method and is not intended to be an exhaustive description of the benefits and methods of the present invention. Rather, it shall be understood that the above process may be more generally used to address a wide variety of quality issues in a wide variety of manners.

Those having ordinary skill in the art will recognize that the present invention includes, among other things, an improved system for determining and addressing a large number of quality issues within a healthcare organization. The present specification shall, therefore, be read and understood to incorporate the uses of the information provided by the present system and method, such as the use described above.

The present system may further include an audit process to ensure the responses on the questionnaire are accurate and consistent. The audit process may include having a redundant questionnaire regarding a specific patient completed by a different person to determine whether the responses on the questionnaire are consistent.

In another embodiment, the audit process may include having a person or entity unrelated to the healthcare organization perform an independent review of a patient's chart and the responses on the questionnaire.

Other methods of auditing responses known in the art are also contemplated as useful in the present auditing step.

In another aspect, the invention is a method of determining a pay-for-performance valuation for a healthcare organization. The above method and system, when utilized, provides an accurate understanding of the quality of the healthcare services and procedures provided by a healthcare organization. Once determined, the quality measures of an organization may be compared to the quality measures of other organizations providing similar healthcare procedures.

As a nonlimiting example, the national benchmark for medication errors, as determined by the ASA is 5.26%. An anesthesia organization, utilizing the present invention, may demonstrate a medication error incidence lower than the industry standard, for example, in 0.02% of their cases. The healthcare organization is able to demonstrate, therefore, improved quality over national standards in relation to medication errors. This improved quality measure, particularly if repeated throughout the organization in relation to other benchmarks, demonstrates the high quality of care provided by that organization. Accordingly, because the healthcare organization is able to accurately demonstrate high quality, that high quality may be used to justify higher fees in a pay-for-performance environment.

Insurance plans, hospitals, Medicare, and Medicaid may be willing to pay more for better quality. Those payment organizations may be more willing to pay these higher fees when the value received by the patients may be clearly demonstrated with accurate quality figures. The ability to quantify superior outcomes and patient satisfaction may result in increased patient referrals and income. Additionally, the healthcare organization may be able to use the data to determine the implementation of a protocol exceeds national benchmarks.

These determinations, and the ability to make them, may further justify the use of the present method and system in pay for performance environments. Accordingly, the present method and system for determining the quality of healthcare procedures provided by a healthcare organization lends itself to the support of a pay-for-performance valuation. The ability of a healthcare organization, such as an anesthesia organization, to implement a process to assure higher quality, efficiency, and patient satisfaction could act as a competitive advantage in attaining new opportunities for business and service.

Moreover, the present method and system, because they include questionnaires and surveys conducted by trained personnel on a large percentage of the patients within a healthcare organization and throughout the continuum of care, lend themselves to accurate and timely reporting that overcomes problems with previous systems. As previously discussed, the present method and system provide accurate and comprehensive quality findings.

Additionally, the use of trained personnel for the completion of the surveys and questionnaires on a real time basis, (i.e., as the procedure is occurring or immediately after the completion of the procedure), results in higher accuracy than surveys solely answered by patients or conducted retrospectively several steps removed from the procedure. The present method, therefore, helps reduce the risk that inaccurate or untruthful answers will be provided by patients in the interest of lowering costs in the pay-for-performance valuation.

In an exemplary embodiment, a smaller number of indicators may be identified as being particularly relevant to a pay for performance valuation. For example, indicators that are specific enough to enable comparison between organizations may be identified as being particularly helpful in determining the pay for performance valuation. In one embodiment, the present survey and/or questionnaire may identify between about 5 and 20 (e.g., 6-10) indicators out of the total list of indicators as being especially helpful in determining the pay for performance valuation. Some exemplary indicators that may be especially useful for determining the pay for performance valuation are one or more of delayed practitioner, perioperative beta blocker protocol used in an at risk patient, Ab given within one hour of incision, wet tap from epidural, awareness under general anesthesia, intraoperative normothermia in colorectal surgery, glucose measured and sliding scale insulin protocol implanted for blood sugar less than 200, regional performed on the wrong location, pneumo/hemothorax from CVL placement, aspiration, pulmonary edema, MI, stroke, implementation of HOB 30° post operatively in mechanically ventilated patients, combinations thereof, and the like.

In one embodiment, the pay-for-performance valuation may be determined by using the quality data collected and analyzed as a part of the present method and system to develop ratings, or scores, for each participant in a healthcare organization. For example, each physician, nurse, technician, or facility, may be individually rated based on the quality measures.

In another embodiment, a physician may be scored and a composite scorecard developed for each practitioner, nurse, or other participant in the healthcare organization based on the performance in several broad categories. Nonlimiting examples of the types of broad categories may include one or more of efficiency measures, practitioner performance, critical quality indicators, patient satisfaction, combinations thereof, and the like. Nonlimiting examples of efficiency measures on which a practitioner may be rated include one or more of percentage of cases delayed, percentage of cases cancelled, percentage of cases that exceed an expected time, combinations thereof, and the like.

Practitioner performance may be measured based on indicators recognized by various associations such as the ASA or other published studies. Similarly, critical quality indicators, such as those discussed above, may be assessed in order to rate the physician in this category. Patient satisfaction may be determined by measuring the overall percent of patient satisfaction and/or scoring the results of the patient survey to determine an overall score. For example, the results could be scored on a scale of 1-5 or on other scales known to those having ordinary skill in the art.

Results in each of the above categories may be determined by utilizing the present system and method. After a practitioner has been rated according to the system, the practitioner's performance may be compared to the performance of other, similarly situated practitioners. Once the comparison has been made, a pay-for-performance valuation for that practitioner may be more accurately and fairly determined than was previously possible under the random sampling quality assessment measurements. Similarly, a scorecard for the entire organization may be developed to compare the organization to other organizations providing similar services.

Similar analyses may also be conducted for other categories, such as facility ratings, procedure and process ratings, nurse or support staff ratings, combinations thereof, and the like. Accordingly, the present system and method provide a more accurate basis for measuring a pay-for-performance valuation because the data from which the valuation is calculated is more comprehensive and accurate than the data provided by the previous quality assessment systems.

Moreover, the improved accuracy of the present quality assessment system and method results in improved accuracy of the pay-for-performance valuations. Insurance plans, hospitals, Medicare, and Medicaid may be more likely to participate in a pay-for-performance environment that is based on such accurate results, because there is assurance that the fee scale is fair and accurate.

Additionally, physicians and healthcare organizations may be more willing to participate in pay-for-performance valuations based on the present system and method because they are assured that the payment received is fair and based on performance, rather than a flat fee being paid to any physician regardless of quality.

Consumers may also be more willing to participate in a pay-for-performance valuation based on the present system and method as a result of the assurance that they are paying fairly for services that are assured of being high in quality.

The quality assessment system and method also allows the consumers to make more educated choices when choosing a physician, healthcare organization, and/or hospital for any type of healthcare procedure. Quality ratings, accurately determined according to the present invention, could be posted along with physician information in the guides listing participating physicians provided by insurance companies, Medicare, and Medicaid. Consumer confidence in their physicians could also be increased through use of the present invention.

In the specification and drawings, there have been disclosed typical embodiments of the invention and, although specific terms have been employed, they have been used in a generic and descriptive sense only and not for purposes of limitation, the scope of the invention being set forth in the following claims.

1. A method for improving the quality of healthcare procedures, the method comprising:
conducting at least a first survey, throughout the continuum of care, of of a plurality of patients of a healthcare organization regarding care provided before, during, and after a procedure, wherein the survey focuses on indicators identified as being relevant to quality of care;
entering responses from the first survey into a database configured to store the responses;
reviewing the responses from the first survey to determine the quality of care provided before, during, and after the procedure;
identifying incidences of poor quality of care, if any;
comparing the responses of the first survey with responses of surveys taken from other patients to recognize patterns of poor quality of care; and
addressing the incidences of poor quality of care by developing methods to address the recognized patterns of poor quality of care.

2. The method of claim 1 wherein the step of conducting at least a first survey comprises collecting information on indicators related to quality of care.

3. The method of claim 2 wherein the step of collecting information on indicators relating to quality of care comprises collecting information on greater than 30 indicators related to the quality of anesthesia care.

4. The method of claim 2 wherein the step of collecting information on indicators relating to quality of care comprises collecting information on greater than 40 indicators related to the quality of anesthesia care.

5. The method of claim 2 wherein the step of collecting information on indicators relating to quality of care comprises collecting information on greater than 50 indicators related to the quality of anesthesia care.

6. The method of claim 1 wherein the step of conducting at least a first survey of patients comprises conducting the survey in person.

7. The method of claim 1 wherein the step of conducting at least a first survey of patients comprises conducting the survey with the use of a computer-based program.

8. The method of claim 1 wherein the step of conducting at least a first survey of patients comprises conducting a survey after a procedure and over the telephone.

9. The method of claim 1 wherein the step of conducting at least a first survey of patients comprises conducting the survey by mail.

10. The method of claim 1 wherein the step of conducting at least a first survey of patients comprises conducting the survey over the Internet.

11. The method of claim 1 further comprising conducting a second survey after the medical procedure is completed but before the patient leaves the healthcare organziation.

12. The method of claim 1 wherein the step of conducting a first survey comprises conducting at least a first survey of substantially all of the patients in a healthcare program.

13. The method of claim 1 wherein the step of conducting a first survey comprises conducting at least a first survey of greater than about 75% of the patients in a healthcare program.

14. The method of claim 1 wherein the step of conducting a first survey comprises conducting at least a first survey of greater than about 50% of the patients in a healthcare program.

15. The method of claim 1 wherein the step of identifying incidences of poor quality of care comprises identifying those indicators specific to practitioner performance versus process performance and communicating in a timely fashion the responses to the first survey to the practitioner.

16. The method of claim 1 wherein the step of reviewing the responses on the first survey to determine the quality of care provided during the procedure comprises having a committee including medical personnel.

17. The method of claim 1 further comprising an additional reviewing step, wherein the responses on the surveys are reviewed by a quality control committee charged with recognizing incidences of repeated poor quality performance.

18. The method of claim 1 wherein the step of identifying incidences of poor quality of care further includes tracking the responses to assess individual practitioner improvement or decline in performance and group improvement or decline in performance.

19. The method of claim 1 wherein the step of comparing the responses on the first survey with results of surveys taken from other patients comprises comparing the responses on the first survey with the responses on surveys taken from other patients having procedures at the same facility as the first patient.

20. The method of claim 1 wherein the step of comparing the responses on the first survey with responses on surveys taken from other patients comprises comparing the responses on the first survey with the responses on surveys taken from other patients undergoing similar procedures as the first patient.

21. The method of claim 1 wherein the step of comparing the responses on the first survey with responses on surveys taken from other patients comprises comparing the responses on the first survey with the responses on surveys taken from other patients having procedures conducted by the same medical personnel as the first patient.

22. The method of claim 1 further comprising determining a pay-for-performance valuation based on the responses on the surveys.

23. The method of claim 1 wherein the step of identifying incidences of poor quality of care comprises identifying medical personnel that have increased incidences of poor quality of care.

24. The method of claim 1 wherein the step of identifying incidences of poor quality of care comprises identifying procedures that have increased incidences of poor quality of care.

25. The method of claim 1 wherein the step of identifying incidences of poor quality of care comprises identifying facilities that have increased incidences of poor quality of care.

26. The method of claim 1 further comprising the step of developing a pay for performance valuation based on the identified incidences of poor quality of care.

27. A system for improving the quality of healthcare procedures, the system comprising:
at least one questionnaire, including a list of indicators recognized as relating to the quality of healthcare, to be completed with respect to a patient's experience before, during, and after undergoing a medical procedure;
a database for storing the responses provided on the at least one questionnaire;
means for reviewing the responses to recognize incidences of poor quality of care before, during, or after a procedure based on the responses on the at least one questionnaire; and
means for controlling quality of care that recognize patterns of poor quality of healthcare based on the responses on the questionnaire and institutes corrections in response to said recognized patterns.

28. The system of claim 26 wherein said at least one questionnaire is specific to said medical procedure.

29. The system of claim 26 wherein said indicators are one or more of case cancellation indicators, case delay indicators, airway and respiratory system indicators, cardiovascular indicators, medication related indicators, neurological block related indicators, critical incident indicators, and post-operative indicators.

30. The system of claim 28 wherein said case cancellation indicators are one or more of pre-operative order violations, abnormal EKG, and abnormal labs.

31. The system of claim 28 wherein said case delay indicators are one or more of pre-operative order violations, delayed surgeon, delayed anesthesiologists, operating room turnover delay, unavailable or delayed lab results, and unavailable or delayed x-rays.

32. The system of claim 28 wherein said airway and respiratory system indicators are one or more of difficult intubations, failed intubation, aspiration, laryngospasm, dental damage or loss, persistent hypoxeia, bronchospasm, ventilator induced injury, and sleep apnea.

33. The system of claim 28 wherein said cardiovascular indicators are one or more of EKG changes, changes to blood pressure, and perioperative beta blocker protocols used in at-risk patients.

34. The system of claim 28 wherein said medication related indicators are one or more of prolonged NM block, the use of an antagonist, and medication errors.

35. The system of claim 28 wherein said neurological block related indicators are one or more of wet tap, spinal requiring mechanical vent and/or intubation, failed labor regional, failed operating regional, nerve injury, and peripheral nerve injury.

36. The system of claim 28 wherein said critical incident indicators are one or more of death, anaphylaxis, aspiration, perioperative bronchospasm, laryngospasm, perioperative hypoxemia, cardiac arrest, blood pressure changes, EKG changes with evidence of ischemia or dysrhythmia required, extended post-anesthesia care unit stay, failed airway, heart attack, pulmonary edema, unanticipated admission to ICU, unplanned reintubation, stroke, wrong site procedure, medication error, transfusion error, nerve injury from position, post-dural headache, and falls.

37. The system of claim 28 wherein said post-operative indicators are one or more of hypothermia, post-operative nausea, post-operative vomiting, problems with pain control, prolonged regional block, prolonged stay in the post-anesthesia care unit, urinary retention, post-dural puncture headache, awareness under general anesthesia, and intra-operative normothermia in colorectal surgery.

38. The system of claim 28 further comprising additional indicators selected from one or more of eye problems, burn injury, pressure injury, medication or fluid extravasations, line complication with central venous line, pneumothorax, hemothorax, hydrothorax, falls, equipment problems, fire, and regionals performed on the wrong location.

39. The system of claim 26 wherein said database is an electronic database.

40. The system of claim 38 further comprising an electronic device for entering responses on said at least one questionnaire into said electronic database.

41. The system of claim 38 wherein said electronic device is one or more of a scanner, a computer, and a personal data assistant.

42. The system of claim 38 further comprising means for auditing the responses to ensure said responses on said questionnaire are accurate.

43. A method of designing a pay-for-performance valuation for a healthcare organization, the method comprising:
conducting at least a first survey of patients regarding care provided before, during, and after a procedure, wherein the survey focuses on indicators identified as being relevant to quality of care;
entering the responses on the first survey into a database configured to store the responses;
reviewing the responses provided in the first survey to determine the quality of care provided during the procedure;
identifying incidences of poor quality of care;
comparing the responses of the first survey with responses of surveys taken from other patients to recognize patterns of poor quality of care; and
scoring the quality of care by comparing the identified patterns of poor quality of care to standards of the industry; and
determining a payment amount based on the scored quality of care.
